(19) United States
(12) Patent Application Publication
(10) Pub. No.: US 2003/0212102 A1
(43) Pub. Date: Nov. 13, 2003

(54) Novel solid dispersion compositions

(76) Inventors: Todd W Koretke, Limerick, PA (US); Kamalnayan H Bhatt, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/297,514

(22) PCT Filed: Jun. 12, 2001
(86) PCT No.: PCT/US01/18853

(51) Int. Cl. A61K 31/47; A61K 9/20

(52) U.S. Cl. 514/312; 424/465

(57) ABSTRACT

This invention relates to novel fast release solid dispersion pharmaceutical compositions with improved solubility and dissolution characteristics, as well as enhanced bioavailability, methods for their preparation and the use of these compositions.

### FIELD OF THE INVENTION

This invention relates to novel fast release solid dispersion pharmaceutical compositions with improved solubility and dissolution characteristics, as well as enhanced bioavailability, methods for their preparation and the use of these compositions. In particular, this invention relates to a solid dispersion pharmaceutical composition consisting essentially of a co-melt of a poloxamer surfactant, a mid-molecular weight polyethylene glycol and a therapeutically active compound that melts without decomposition at a temperature below the flash point of the polyethylene glycol.

### BACKGROUND OF THE INVENTION

A solid dispersion formulation is a drug-containing pharmaceutical bulk substance comprising the drug dissolved or dispersed in a polymer. Solid dispersions are useful for enhancing the solubility of the drug and/or for controlling the rate of release of the drug from a dosage form or improving the bioavailability of drugs. Typically, solid dispersions are slow release or controlled release formulations.

Conventional techniques for producing solid dispersions range from a melt process in which the temperature is above that of the polymer used forming a fine colloid dispersion of drug particles with some solubilization of the drug in the polymer to a co-melt process using a temperature above the polymer and drug in the melt. Often the molten mixture is then cooled rapidly, resulting in a congealed mass which is subsequently milled to produce a powder which is then capsulated or tableted. While seemingly simple, this technique has disadvantages if, for example, the drug and polymer are not miscible in the molten state. In addition, the process is limited in that it tends to lead to drug decomposition due to the high temperatures required to melt the components.

When difficulty arises with thermal instability and/or miscibility between the drug and the carrier, a hybrid method for making solid dispersions, called the fusion-solvent method is utilized. The drug is first dissolved in a small quantity of organic solvent and then added to the molten carrier. The solvent is then evaporated to generate a product that is subsequently milled to produce a powder. However, this solvent process also has disadvantages, e.g., explosion hazard during production, difficulty in removing all traces of solvent from the solid dispersion product for pharmaceutical use, and diffusion of solvent into the atmosphere causing pollution.

Other problems limiting the commercial application of solid dispersion techniques involve, method of preparation, reproducibility of physicochemical properties, formulation of pharmaceutically exceptable dosage forms, the scale up to manufacture GMP clinical supplies, and the physical and chemical stability of the drug and excipients.

In order to overcome the above disadvantages, the art suggests a number of options, e.g., avoiding the co-melt temperatures, and combining a poorly soluble drug with a carrier such as polyvinyl pyrrolidone (PVP) or high molecular weight polyethylene glycol (e.g., PEG 6000), then spraying the drug/carrier mixture with an aqueous mixture of a plasticizer/solubilizer (e.g., low molecular weight PEG 200, 300, 400 or 600, and an optional surfactant such as Tween 80) in a fluid bed granulator, extruding the resulting granulation through a twin-screw extruder with at least one heating zone and milling the extrudate (WO 93/11749, published Jun. 24, 1993). In U.S. Pat. No. 5,456,923, issued Oct. 10, 1995, a twin-screw extruder is employed with pH-dependent polymers, e.g., various derivatives of HPMC. Published international application WO 93/23022, published Nov. 25, 1993, discloses a co-melt combining a drug tebufelone (15-75%) with a poloxamer surfactant having a melting point of 40° C. or greater (25-65%). PEG (0-60%) is disclosed as an optional additional component to the solid dispersion.

The present invention increases the bioavailability of water insoluble drugs through the formation of a fast release solid pharmaceutical dispersion without the need for using organic solvents, without thermal decomposition of the drug at temperatures above the melting point of the drug, and without the need for a milled or otherwise altered solid dispersion.

### SUMMARY OF THE INVENTION

The instant invention relates to pharmaceutical compositions, methods for their preparation and their use, in dosage form, comprising a fast release solid dispersion which is a solidified co-melt mixture containing amorphous drug consisting essentially of:

(a) from about 0.1% to about 20% of drug active;

(b) from about 2% to about 20% of a poloxamer surfactant having an HLB value between about 20 and about 30; and

(c) from about 60% to about 97.9% of a mid-molecular weight polyethylene glycol, wherein the drug melts without decomposition at a temperature below the flash point of polyethylene glycol.

More specifically, this invention relates to a fast release solid dispersion which is a solidified co-melt mixture containing amophous drug consisting essentially of:

(a) from about 10% to about 20% of (S)-(−)-N-(α-Ethylbenzyl)-3-hydroxy-2-phenylquinoline-4-carboxamide;

(b) from about 5% to about 10% of a poloxamer surfactant, preferably Poloxamer 188; and

(c) from about 70% to about 85% of a mid-molecular weight polyethylene glycol.

### DETAILED DESCRIPTION OF THE INVENTION

It has been discovered that a co-melt mixture of a poorly soluble drug with a poloxamer surfactant having an HLB value between about 20 and about 30, in particular Poloxamer 188, and a mid-molecular weight PEG, wherein the drug melts without decomposition at a temperature below the flash point of the polyethylene glycol (approximately 230° C.), provides greatly enhanced solubility, rapid dissolution and bioavailability characteristics. Unexpectedly, the inventive combination demonstrates a nine to ten-fold increase in solubility over the same drug substance alone in one hour of dissolution in USP apparatus 2 non-sink conditions, (i.e., non-sink refers to the solubility limit of the drug in the media, typical sink conditions described by the USP are 3-5 times the less drug concentration than solubility limit). This invention enables the solid dispersion to be a fast-release solid dispersion formulation, whereas typical solid dispersions enhance solubility, and therefore bioavailability, but are slow release formulations.

Without being bound to any particular mechanism of action, the following represents the rationale for the unexpected increase in dissolution rate enabling this invention to provide a fast-release solid dispersion formulation. The interaction of the polymers and the hydrophobic drug are crucial for proper stable solubilization of the otherwise poorly soluble drug substance. The application of solid dispersions for fast-release mechanisms to increase bioavailability relates only with poorly water soluble or poor wetability compounds. These poor dissolution properties normally occur when the compound is highly hydrophobic in nature, thus this theory applies to those compounds.

The polymer polyethylene glycol, is composed of hydrophilic oxyethylene chains. The non-ionic surfactant, poloxamers, are composed of polyoxyethylene-polyoxypropylene copolymers. The polyoxyethylene segment is hydrophilic while the polyoxypropylene segment is hydrophobic. The use of both polymers together allows the interaction of polymers with a hydrophobic drug to enhance the dissolution rate and bioavailability. When in amorphous form, i.e., after a melt above the drug substance melting point is cooled forming a stable solid dispersion, the poloxamer acts as the link between the polyethylene glycol and the drug. The direct result of the interaction is a steady erosion of the solid dispersion allowing the drug to be solubilized without the nucleation of the drug from the amorphous state to form crystals. A diffusion of the polymers too quickly would allow the formation of the drug crystals that were merely left behind by the water-soluble polymers. The relationship between drug, surfactant and polymer is crucial for complete and stable supersaturation of the non-water soluble drug in aqueous media at relevant pH.

Certain quinoline derivatives are known to be neurokinin-3 receptor antagonists, and thereby effective in treating certain disorders, in particular Chronic Obstructive Pulmonary Disorder (COPD) and urinary incontinence. Of particular interest with respect to this invention is (S)-(−)-N-(α-Ethylbenzyl)-3-hydroxy-2-phenylquinoline-4-carboxamide, disclosed in WO 95/32948, published Dec. 7, 1995, as Farina et al. Related compounds are disclosed in WO 96/02509, published Feb. 1, 1996, as Farina et al.; WO 97/19926, published Jun. 5, 1997, as Giardina et al.; WO 97/21680, published Jun. 19, 1997, as Giardina et al.; WO 98/52942, published Nov. 26, 1998, as Giardina et al., WO 98/05640, published Feb. 12, 1998, as Grugni et al., and EP0673928, published Sep. 27, 1995, as Bichon et al., which are incorporated herein by reference in their entirety.

Further to the quinoline derivatives described therein, the invention is useful for any poorly water soluble, poorly wetable compound that melts without decomposition below the flash point of polyethylene glycol.

This invention involves chemically stable pharmaceutical compositions intended for peroral administration to mammals, particularly humans. The instant compositions consist essentially of a mid-molecular weight PEG, a drug which melts without decomposition at a temperature below the flashpoint of the PEG, and a poloxamer surfactant with an HLB value between about 20 and about 30, in particular, Poloxamer 188 (with the tradename Pluronic® F68) commercially available from BASF.

The term “solid dispersion” is used herein at all occurrences to mean a material which is solid at room temperature, and which has been produced by blending melted drug with the surfactant and the PEG, whereby a homogeneous melt mixture results, and cooling the resultant mixture so that it forms a solid with the components substantially uniformly dispersed therein.

More in particular, the fast release solid dispersions of this invention consist essentially of about 0.1% to 20% drug; about 2% to about 20% of a surfactant having an HLB value between about 20 and about 30; and about 60% to about 97.9% PEG. A preferred embodiment of the invention consists essentially of about 10% to about 20% drug; about 5% to about 10% of a surfactant having an HLB value between about 20 and about 30, preferably Poloxamer 188; and from about 70% to about 85% of a mid-molecular weight polyethylene glycol. The drug should melt without decomposition at a temperature below the flashpoint of the PEG. For the free base form of the preferred active compound useful in this invention, (S)-(−)N-(α-Ethylbenzyl)-3-hydroxy-2-phenylquinoline-4-carboxamide, the melting point is 165° C.

The free base form of (S)-(−)-N-(α-Ethylbenzyl)-3-hydroxy-2-phenylquinoline-4-carboxamide has very poor in vitro solubility and dissolution characteristics (31 micrograms/mL in simulated gastric fluid without enzyme, pH 1.2). This typically translates into poor bioavailability. The solid dispersions of mid-molecular weight (1500-6000) PEG's and (S)-(−)-N-(α-Ethylbenzyl)-3-hydroxy-2-phenylquinoline-4-carboxamide had an increased solubility and dissolution rate at 37° C. in simulated gastric fluid (“SGF”) without enzyme. While enhanced solubility might be expected with the addition of PEG and a surfactant, an unexpected nine- to ten-fold increase in solubility was observed with the triple combination of the instant inventive formulation. Therefore, in addition to being more soluble and bioavailable, the instant formulation is also surprisingly fast release.

PEG's of average molecular weight (“MW”) below 800 are characteristically liquid at room temperature and very hygroscopic, thus not suitable for dispersions. Solid PEG's below MW 2000 are waxy solids with low melting points and some hygroscopicity, thus manufacturability and stability are poor. Mid-MW PEG's from 2400 to 8000 are relatively stable with fair processability, but MWs above 4600 are very viscous liquids when melted having poor flowability and mixing. Higher MW PEG's (such as 20,000) would also be unsuitable for use in solid dispersions because their slower dissolution time would not be suitable for a fast release mechanism. Higher amounts of peroxides may also be trapped in the folded crystalline structure of the high MW PEG's thus creating chemical stability problems.

Suitable polyethylene glycols include mid-molecular weight PEG's with an average molecular weight ranging from 1500 to 6000, preferably from 3000 to 6000. Particularly preferred PEG's useful in the solid dispersions of the instant invention are PEG 3350 (also referred to as Carbowax™Sentry™Polyethylene Glycol 3350 Powder NF,FCC), and PEG 6000, available from Union Carbide Corporation, Danbury, Conn. The flash point of PEG 3350 is 246° C. (Pensky-Martens closed cup ASTM D 93) or 279° C. (Cleveland open cup ASTM D 92). While the flash point of PEG's are based upon a molecular weight range associated with the particular PEG (e.g., PEG is specific to a range of PEG polymers of molucular weight range 3000 to 3700) and therefore, the flashpoint may vary depending on the quality of the material used, the flash point of PEG 6000 is about 246° C.

Examples of preferred poloxamer surfactants useful in the solid dispersions of this invention include Poloxamer 188 (Pluronic® F68) commercially available from BASF Corporation, New Jersey. Poloxamer 188 is about 80% by weight poly(oxyethylene), with an average molecular weight of between about 7680 and about 9510, and a melting point of about 52° C.

Suitably, the solid dispersions of this invention may contain up to about 10% inert fillers that do not materially effect the properties of the end product. Examples of such fillers include, hydroxypropylmethylcellulose phthalate 22084 (HP50), hydroxypropylmethylcellulose phthalate 220731 (HP55), hydroxypropylmethylcellulose acetate succinate (AQOAT), carboxymethyl-ethylcellulose (CMEC), cellulose acetate phthalate (CAP), methacrylic copolymer LD (L30 D55), methacrylic copolymers S (S-100), aminoalkyl methacrylate copolymer E (gastric coating base), poly(vinyl acetal) diethylaminoacetate (AEA), polyvinylpyrrolidone (K-25, 30, 90; PVP), ethylcellulose (EC), methacrylic copolymer RS (RS 30D), polyvinyl alcohol (PVA), methylcellulose (MC), hydroxypropylcellulose (HPC), hydroxypropylmethylcellulose 2208 (Metolose 90SH), hydroxypropylmethylcellulose 2906 (Metolose 65SH), hydroxypropylmethylcellulose 2910 (Metolose 60SH), carboxymethylcellulose sodium (sodium cellulose glycolate), dextrin, pullulan, Acacia, tragacanth, sodium alginate, propylene glycol alginate, agar powder, gelatin, starch, processed starch, phospholipids (lecithin), glucomannan and the like.

The fast release solid dispersions of this invention are preferably made by melting the drug, the polyethylene glycol and the poloxamer surfactant together, with mixing, to form a homogeneous melt mixture. The tertiary melt mixture is then cooled rapidly to solidification. Suitably, other components may be added to the tertiary mixture prior to solidfication.

Preferred dosage form compositions of the instant invention are made from the above solid dispersions. Preferred solid dispersions of this invention may be filled into capsules or molds prior to solidfication. Alteration of the solid dispersion by physical means (i.e., additional energy added) from the original cooled solid form yielded drastically different solubilization due to uncontrolled erosion rate and nucleation of the drug substance in the milled high surface area formulation. This property distinguishes this invention from known solid dispersion dosage forms in which solid dispersion of drug and PEG were milled and filled into capsules or tableted.

The drug to PEG ratio effects dissolution since higher drug loads decrease dissolution rate. The ratio of poloxamer to PEG is crucial to solid dispersion erosion rate stability. It has been found that a preferred ratio of components for the instant invention is 4 parts drug:1 part poloxamer surfactant:15 parts mid-molecular weight polyethylene glycol.

### EXAMPLES

Preparation of the Solid Dispersion:

Solid dispersions were formed by the melt or fusion method of manufacture. The melts were made in a Digi-Block heater with aluminum heating blocks using 13×100 mm borosilicate glass tubes or 22 to 44 mL borosilicate scintillation vials. Melt temperatures were above that of the melting point of the free base drug component, 165° C., although most dispersions at low drug composition would be completely melted at approximately 152° C. The block temperature was controlled by the blocks calibrated thermocouple and control program. The temperature was confirmed by a calibrated thermocouple and calibrated thermometer. Most carriers studied had melting temperatures of approximately 50° C. attributing to the lower melting temperatures of the matrix at high polymer composition.

After 5 to 20 minutes of melt time the molten matrix was pipetted into size 0 or smaller gelatin or hydroxypropylmethyl cellulose (HPMC) capsules. The matrix temperature was above 150° C. during filling, but deformation of the gelatin capsules was not prevalent due to the rapid cooling of the dispersion at the interface with the capsule at room temperature. The dispersions were allowed to cool and harden overnight in a desiccator at room temperature or

5° C. Some dispersions were poured into teflon weigh dishes that allowed easy recovery of the solid dispersion for alternate investigation.

Physical matrix mixtures and melted carriers were made and tested to compare the baseline solubility and background absorbance to that of the solid dispersions. The resultant solid dispersion were yellow-orange hard solids with some air pockets formed by the cooling process. The dispersion solubility and dissolution rate were analyzed by USP Apparatus 2 dissolution. The dissolution was performed under the following conditions: SGF dissolution media, 0.1 M HCL without enzymes pH 1.2; non-sink conditions of 350-370 mL media; paddle speed of 50 rpm; temperature of 37° C.; 10 mL sample pull not replaced. Analysis of the (S)-(−)-N-(α-Ethylbenzyl)-3-hydroxy-2-phenylquinoline-4-carboxamide was performed using a UV spectrophotometer at 359 nm compared to standard solutions.

A maximum temperature of 165° C. was necessary to assure that the drug was completely changed to the amorphous form.

All publications, including, but not limited to, patents and patent applications cited in this specification, are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration it is believed that one skilled in the art can, given the preceding description, utilize the present invention to its fullest extent. Therefore any examples are to be construed as merely illustrative and not a limitation on the scope of the present invention in any way. The embodiments of the invention in which an exclusive property or privilege is claimed are defined as follows.

What is claimed is:

1. A fast release pharmaceutical composition consisting essentially of a poloxamer surfactant, a mid-molecular weight polyethylene glycol and an active compound that melts without decomposition at a temperature below the flash point of the polyethylene glycol.

2. A fast release pharmaceutical composition consisting essentially of a co-melt of a poloxamer surfactant, a mid-molecular weight polyethylene glycol and an active compound that melts without decomposition at a temperature below the flash point of the polyethylene glycol.

3. A fast release solid dispersion co-melt composition consisting essentially of a poloxamer surfactant, a mid-molecular weight polyethylene glycol and an active compound that melts without decomposition at a temperature below the flash point of the polyethylene glycol.

4. A fast release solid dispersion composition which is a solidified co-melt mixture consisting essentially of:
(a) from about 0.1% to about 20% of drug active;
(b) from about 2% to about 20% of a poloxamer surfactant having an HLB value between about 20 and about 30; and
(c) from about 60% to about 97.9% of a mid-molecular weight polyethylene glycol, wherein the drug melts without decomposition at a temperature below the flash point of polyethylene glycol.

5. The composition of any of claims 1, 2, 3 or 4, wherein the active compound is (S)-(−)-N-(α-Ethylbenzyl)-3-hydroxy-2-phenylquinoline-4-carboxamide.

6. A method for preparing the composition according to claims 1, 2, 3 or 4, comprising:
(a) melting the drug, the polyethylene glycol and the poloxamer surfactant together, with mixing, to form a tertiary homogeneous melt mixture;
(b) cooling the melt mixture until solidified; and
(c) forming a preferred dosage form from the solidified melt mixture.

7. The composition of any of claims 1, 2, 3 or 4, wherein the surfactant is a poloxamer surfactant.

8. The composition of claim 7, wherein the poloxamer surfactant is Poloxamer 188.

9. The composition of any of claims 1, 2, 3 or 4, wherein the polyethylene glycol has an average MW between about 1500 and 6000.

10. The composition of claim 9 wherein the polyethylene glycol has an average MW between about 3000 and 6000.

11. The composition of claim 9 wherein the polyethylene glycol is selected from PEG 3350 or PEG 6000.

12. A method for delivering an active to a mammal in need of such active which comprises orally administering a therapeutically effective amount of the composition according to claims 1, 2, 3 or 4.

13. The method of claim 12 wherein said composition is administered to a mammal exhibiting symptoms of COPD or urinary incontinence.

14. A fast release solid dispersion composition which is a solidified co-melt mixture consisting essentially of:
(a) from about 10% to about 20% of (S)-(−)-N-(α-Ethylbenzyl)-3-hydroxy-2-phenylquinoline-4-carboxamide;
(b) from about 5% to about 10% of a poloxamer surfactant; and
(c) from about 70% to about 85% of a mid-molecular weight polyethylene glycol, wherein the drug melts without decomposition at a temperature below the flash point of polyethylene glycol.

15. The composition of any of claims 1, 2, 3 or 4, wherein the ratio of drug:poloxamer surfactant:polyethylene glycol is 4 parts drug:1 part poloxamer surfactant:15 parts polyethylene glycol.

16. A fast release solid dispersion composition which is a solidified co-melt mixture containing amorphous drug consisting essentially of:
(a) from about 0.1% to about 20% of drug active;
(b) from about 2% to about 20% of a poloxamer surfactant having an HLB value between about 20 and about 30; and
(c) from about 60% to about 97.9% of a mid-molecular weight polyethylene glycol, wherein the drug melts without decomposition at a temperature below the flash point of polyethylene glycol.

17. The composition of claim 16, wherein the solidified co-melt mixture containing amophous drug consists essentially of:
(a) from about 10% to about 20% of (S)-(−)-N-(α-Ethylbenzyl)-3-hydroxy-2-phenylquinoline-4-carboxamide;
(b) from about 5% to about 10% of a poloxamer surfactant, preferably Poloxamer 188; and
(c) from about 70% to about 85% of a mid-molecular weight polyethylene glycol.
